(19) United States
(12) Patent Application Publication
(10) Pub. No.: US 2009/0042211 A1
(43) Pub. Date: Feb. 12, 2009

(54) Method for the Selective Detection of Pathological Protein Depositions

(76) Inventors: Eva Birkmann, Korschenbroich (DE); Detlev Riesner, Dusseldorf (DE); Nicole Weinmann, Aurich (DE); Oliver Schafer, Essen (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/994,727

(22) PCT Filed: Jul. 4, 2006
(86) PCT No.: PCT/EP2006/006512

§ 371 Date: Aug. 6, 2008

(30) Foreign Application Priority Data
Jul. 4, 2005 (DE) 102005031429.5

(51) Int. Cl. G01N 33/53; G01N 33/566

(52) U.S. Cl. 435/7.1; 436/501

(57) ABSTRACT

The invention relates to a method for the selective detection of the presence and/or quantity of pathological protein depositions.

The invention relates to a process for the selective determination of the presence and/or amount of pathological protein deposits, especially those protein deposits that are associated with neurodegenerative diseases. The process comprises: (a) immobilizing on a surface a capture molecule having specific binding affinity for substructures of said protein deposits; (b) contacting the immobilized capture molecule with a sample to be measured that is suspected of containing pathological protein deposits or substructures thereof; (c) incubating the preparation to allow a complex to be formed from the immobilized capture molecule and said substructures of said protein deposits; (d) contacting the resulting complex with at least one detectable unit having specific binding affinity for said substructures of the protein deposits and producing an optically detectable signal, wherein at least one of said at least one detectable unit produces a signal detectable by means of spectroscopic methods; and (e) detecting the complex formation by measuring the overall signal produced by said at least one detectable unit.

A number of diseases is associated with the occurrence of protein deposits. However, to date, it has been essentially unclear if such protein deposits are only a manifestation of the respective clinical picture, or if they are actually causally responsible for the etiology and/or progression of such diseases. Thus, for example, neurodegenerative diseases are known in which protein deposits referred to as amyloid plaques occur in the brain of afflicted subjects. Such diseases include, inter alia, Alzheimer's disease, Parkinson's disease, Huntington's chorea, hereditary cerebral amyloid angiopathy and the transmissible spongiform encephalopathies. The latter include, for example, Creutzfeldt-Jakob disease (CJD) in humans, scrapie in sheep or bovine spongiform encephalopathy (BSE) in cattle as well as other syndromes, formerly referred to as “slow virus” diseases, such as Kuru.

Today, these diseases are summarized under the term of “prion diseases” (surveys in Prusiner, S. B. (1982) Science 216, 136-144; Weissmann, C. (1996) FEBS Lett. 389, 3-11; Riesner, D. (1996) Chemie in unserer Zeit, pp. 66-74; Prusiner, S. B. (1998) Proc. Natl. Acad. Sci. USA 95, 13363-13383).

However, pathological protein deposits not only appear in diseases of the neuronal system, but are also observed in other organs. For example, in type II diabetes mellitus, a diabetic nephropathy is observed in some patients, and a matrix disturbance from protein deposits is discussed as the reason thereof. A survey of non-neuronal diseases accompanied with the formation of pathological protein deposits is found in Sipe, W. (1992) Annu. Rev. Biochem. 61, 947-975.

In the transmissible spongiform encephalopathies, deposits of the infectious form of the prion protein (PrP^{Sc}) are believed to be causally related with the pathogenesis. This modified prion protein is capable of interacting with the normal cellular form PrP in such a way that the infectious form PrP^{Sc} causes a conformational change of the wild type form PrP to the infectious form. The infectious forms PrP^{Sc} aggregate and form the pathological protein deposits characteristic of the indication.

In recent years, it has been mainly bovine spongiform encephalopathy (BSE) that reached the awareness of the public, especially because BSE is associated with the human Creutzfeldt-Jakob disease. Therefore, establishing detection methods for the diagnosis of prion diseases is of particular importance. For example, from a veterinary point of view, it is necessary to ensure that contaminated products, for example, meat, of BSE-infected cattle or of scrapie-infected sheep do not come into circulation. In addition to a reliable and quick diagnosis, a high sensitivity of the detection is desirable, so that infected animals can be recognized early. Further, the preparation of the biological sample material should require as little an expenditure of work as possible in order to enable serial tests to be efficiently performed. For a reliable early detection of prion diseases, it is further necessary to avoid losses of pathogenic material in the preparation of the sample material. Current BSE test methods (see, e.g., Hörnlimann, B. et al. (2001) Prionen und Prionkrankheiten, Gruyter, 290-295; Pitschke, M. et al. (1998) Nat. Med. 4, 832-834; Safar, J. G. et al. (1998) Nat. Med. 4, 1157-1165; Bieschke, J. et al. (2000) Proc. Natl. Acad. Sci. USA 97, 5468-5473; Safar, J. G. et al. (2002) Nat. Biotechnol. 20, 1147-1150; Thomzig, A. et al. (2004) J. Biol. Chem. 279, 33847-33854) utilize the proteinase K (PK) resistance of infectious PrP^{Sc} as an essential criterion. However, the proportion of PK-resistant PrP^{Sc} may vary quite highly both among individuals and among different areas of one organ from the same individual. In addition, the proportion of PK-resistant PrP^{Sc} in BSE-infected cattle is lower as compared to scrapie-infected sheep. However, this means that a treatment of the sample material with proteinase K may result to a substantial bias in the measuring results because portions of the pathogenic material are not covered. Therefore, it is desirable for a sensitive detection method to measure the infectious prion proteins or their mutual association directly.

Further, it is unknown what ratio of PK-sensitive to PK-resistant PrP^{Sc} is found in early stages of the disease.

A further challenge in connection with infection control of prion diseases resides in the fact that there is an at least hypothetical risk that, for example, BSE-infected material may be transmitted to other species, such as sheep, through contaminated animal feed (including meat, meat meal or bone meal). However, the clinical symptoms of scrapie cannot be distinguished with sufficient precision from those of BSE experimentally induced in sheep (Bradley, R. (2004) Prions: A Challenge for Science, Medicine, and the Public Health System (Rabenau, H. F., Cinatl, J., and Doerr, H. W., Editors) S. Karger AG, Basel, Switzerland, pp. 146-185). To date, a time-consuming and cost-intensive typing in mouse models has been necessary for this purpose. Therefore, there is a need for methods by which a prion protein from a particular species can be selectively detected.

The corresponding requirements to a detection method for pathological protein deposits are not limited to the detection of prions and can be transferred to the detection of further diseases associated with protein deposits.

European Patent EP 1 015 888 discloses a process which directly measures the association of substructures of the protein deposits as a target to a probe capable of associating with the target. A “substructure” is intended to mean a pathological protein as such or an aggregation of several proteins that includes pathological proteins. The interaction of such a substructure with a probe is preferably detected by spectroscopy, wherein various structures capable of interacting with the substructure can be employed as the probe. Consequently, therefore, due to the self-aggregation of prion proteins, the pathological protein itself may also serve as a probe in this case. The protein aggregates can be directly detected due to intrinsic properties of the respective molecules, or are detected indirectly by the association of, for example, fluorescence-labeled antibodies and/or fluorescent synthetically prepared probe molecules and excitation of such fluorescent compounds by laser light.

However, by analogy with the above mentioned other current detection methods, the process disclosed in EP 1 015 888 has the drawback that the labeled targets, such as infectious prion aggregates, are measured while freely mobile in solution. Quite usually, the aggregates are in a very low concentration and unequally distributed in the sample to be measured. For example, larger aggregates will sink faster and thus disappear from the measurable volume. Thus, this fact causes measuring errors, which are in part substantial.

Therefore, it is the object of the present invention to provide an improved process for detecting pathological protein deposits that, as compared to the known detecting methods, not only has a higher measuring accuracy in connection with an increased sensitivity, but allows the selective determination of a specific protein deposit.

This object is achieved by a process for the selective determination of the presence and/or amount of pathological protein deposits according to independent claim 1, comprising:
(a) immobilizing on a surface a capture molecule having specific binding affinity for substructures of the protein deposits to be determined;
(b) contacting the immobilized capture molecule with a sample to be measured that is suspected of containing pathological protein deposits or substructures thereof;
(c) incubating the preparation to allow a complex to be formed from the immobilized capture molecule and said substructures of the protein deposits to be determined;
(d) contacting the resulting complex with at least one detectable unit having specific binding affinity for said substructures of the protein deposits to be determined and producing an optically detectable signal, wherein at least one of said at least one detectable unit produces a signal detectable by means of spectroscopic methods; and
(e) detecting the complex formation by measuring the overall signal produced by said at least one detectable unit.

The invention is based on the surprising finding that a selective and efficient detection process for protein deposits with high sensitivity that overcomes all the above mentioned drawbacks of the previously known methods could be established by immobilizing the protein deposit to be determined on a surface in combination with the use of specific capture molecules and detection units.

The process according to the invention is characterized by the immobilization on a surface of the protein deposits to be determined. This results in a concentration of the protein aggregates within the surface that causes a substantial increase of test sensitivity. At the same time, it is possible to scan the whole surface and thus to detect all the immobilized protein aggregates, especially singly as well, and to count them, whereas in a three-dimensional measurement in solution, frequently only a partial volume is analyzed and thus a substantial proportion of the protein aggregates present are not covered. In addition, the sinking of larger protein aggregates out of the measuring area is prevented. Both aspects cause a significant improvement of the measuring accuracy.

The protein deposits can be immobilized on any surface, for example, on a glass surface, a plastic surface or a metal surface. Preferably, the protein aggregates are immobilized on an analytical or assay chip. Such chips are commercially available from numerous suppliers.

The immobilization to the surface is effected through a capture molecule that has specific binding affinity for substructures of the protein deposits to be determined, i.e., that binds such substructures with a clearly higher affinity even in comparison with similar or homologous substructures. In other words, the capture molecule distinguishes between similar structures, whereby the specificity and selectivity of the detection is further increased.

The capture molecules are covalently or non-covalently bonded to the surface. In particular embodiments of the invention, the surface is activated before the protein aggregates are immobilized. Such activation can be achieved, for example, by flaming the surface and coating it with various polymers, for example, with poly-D-lysine.

“Substructures” of the protein deposits to be determined for which the capture molecules have a specific binding activity is intended to mean monomeric or oligomeric units of the protein deposits, for example, monomeric prion proteins or oligomeric protein aggregates. However, a “substructure” according to the invention may also be part of a monomer, for example, a peptide.

Preferred are capture molecules selected from the group consisting of monoclonal antibodies, polyclonal antibodies and antibody fragments, monoclonal antibodies being particularly preferred.

By using specific monoclonal antibodies, scrapie-specific or BSE-specific prion proteins, for example, can each be selectively immobilized to a surface and detected within the same sample to be measured.

In other embodiments, the capture molecule itself also consists of substructures of pathological protein deposits or fragments thereof, wherein such substructures may be of natural origin or have been prepared recombinantly. Due to the self-aggregation, such as of prion proteins, the protein deposits to be detected are also immobilized on the surface. In this case, both substructures of the protein deposit to be detected (homologous system) and substructures of other protein deposits (heterologous system) may be used as the capture molecule. For example, a substructure derived from amyloid plaques of a BSE can be used as a capture molecule for the detection of substructures from tissue afflicted with Alzheimer's disease.

Optionally, free binding sites remaining on the surface after the immobilization of the capture molecule may be blocked by incubation with a blocking reagent in order to reduce the non-specific binding of the protein aggregates to be determined. For example, bovine serum albumin (BSA) solution or fat-free skimmed milk may be used as blocking reagents.

Although not restricted thereto, the protein deposits to be determined qualitatively and/or quantitatively are preferably associated with neurodegenerative diseases. In particular embodiments of the invention, the neurodegenerative diseases are selected from the group consisting of transmissible spongiform encephalopathies, Alzheimer's disease, Parkinson's disease, Huntington's chorea and hereditary cerebral amyloid angiopathy.

In preferred embodiments of the invention, the protein deposits to be detected are associated with transmissible spongiform encephalopathies, Creutzfeldt-Jakob disease (CJD), scrapie and bovine spongiform encephalopathy being particularly preferred.

The protein deposits to be determined can be detected in any biological sample to be measured, which may be derived, for example, from a body fluid or a tissue. In particular embodiments of the invention, the body fluid is selected from the group consisting of cerebrospinal fluid, lymph, blood, urine and sputum, cerebrospinal fluid and blood being particularly preferred. In other preferred embodiments, the sample to be measured is derived from brain tissue.

In other preferred embodiments, the sample to be measured, before being contacted with the capture molecule, is subjected to a purification method in order to isolate the protein deposits to be determined from any contaminants. Depending on the type of sample, the supposed concentration of the protein deposits to be determined, the detection method employed and the like, a partial or (almost) complete purification/isolation can be performed. The samples may be treated with physical and/or chemical standard methods (e.g., ultrasound, temperature changes, incubation with solutions of different ionic strengths, chaotropic salts, surfactants and enzymes). Such methods may be applied singly of in any combination desired.

In preferred embodiments of the invention, the samples to be measured are purified by sodium phosphotungstate (NaPTA) precipitation (Safar, J. D. et al. (1998) Nat. Med. 4, 1157-1165), dispensing with the addition of proteases, especially proteinase K.

According to the invention, the sample to be measured is subsequently incubated with the immobilized capture molecule to enable the formation of a complex from the immobilized capture molecule and the substructures of the protein deposits to be determined.

The detection of the complex formation between the capture molecule and the substructure to be detected is effected by contacting the complex with at least one detectable unit. A “detectable unit” is intended to mean a probe molecule that has a specific binding affinity for the substructures of the protein deposits to be determined and produces an optically detectable signal, wherein said optically detectable signal may be produced by the probe molecule itself or by a binding partner coupled to said probe molecule. Examples of such binding partners are radioactive fluorescent, chemiluminescent or bioluminescent labels as well as metal particles (e.g., colloidal gold). At least one of said at least one detectable unit produces a signal detectable by means of spectroscopic methods.

In preferred embodiments of the invention, two detectable units are contacted with the complex simultaneously. If two or more detectable units are used, the contacting with the complex can be effected simultaneously or successively.

In preferred embodiments of the invention, said at least one detectable unit comprises a protein or polypeptide, wherein such protein or polypeptide may be of natural origin or have been prepared recombinantly. In particularly preferred embodiments, said protein or polypeptide is selected from the group consisting of monoclonal antibodies, polyclonal antibodies and antibody fragments, monoclonal antibodies being preferred. However, the detectable units may also be, for example, small organic molecules, nucleic acids (single- or double-stranded DNA or RNA) or polysaccharides.

Also preferred are embodiments of the invention in which the optically detectable signal produced by said at least one detectable unit is selected from the group consisting of absorption, fluorescence, chemiluminescence and bioluminescence emissions.

In particularly preferred embodiments, the detectable units consist of fluorescence-labeled antibodies, especially monoclonal antibodies.

Optionally, after the detectable units have been contacted with the complex, one or more washing steps may be performed in order to remove unbound detectable units, which may otherwise interfere with the measuring result.

The complex formation is detected by measuring the overall signal produced by said at least one detectable unit. The term “overall signal produced” means that, when more than one detectable unit is used, the individual optically detectable signals produced are detected (coincidence measurement), correlated and evaluated. This increases the specificity of the detection, because positive signals occur only if the various detectable units are simultaneously bound to the complex to be detected.

Preferably, the protein deposits to be determined are detected by means of spectroscopic detection methods, such as confocal fluorescence spectroscopy, fluorescence correlation spectroscopy (FCS), FCS in combination with cross-correlation and single particle immunosorbent laser-scanning assay.

In this method, a laser beam excites the protein complexes to be detected to emit a strong fluorescent light, which is recorded by means of a detector, preferably a confocal optical system. Due to the optical properties of the system, detection of single molecules is enabled, so that individual protein aggregates can be counted. As mentioned above, the sensitivity and specificity of the detection can be additionally enhanced by a cross-correlation, for example, by using two different monoclonal antibodies labeled with different fluorescent dyes and determining the simultaneous binding by a cross-correlation of the signals.

These spectroscopic detection methods are respectively applied in connection with corresponding evaluation methods, for example, fluorescence intensity distribution analysis.

The immobilization on a surface of the protein deposits to be determined, in contrast to determination in solution, enables the overall signal produced by said at least one detectable unit to be measured by scanning the surface, for example, by means of a single particle immunosorbent laser-scanning assay, wherein a plurality of adjacent subareas of the surface area are scanned, and the individual values subsequently added.

The invention is further illustrated by the following non-limiting Figures and Examples.

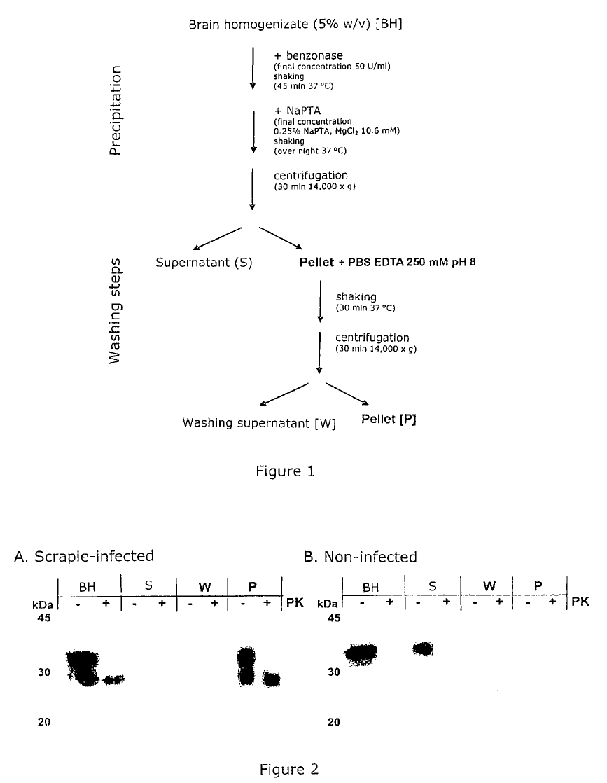

FIG. 1 shows a schematic representation of the protocol used for the purification of PrP^{Sc} from hamster brain tissue by means of NaPTA precipitation without adding proteinase K.

FIG. 2 shows a Western blot analysis of the individual steps of the NaPTA precipitation for the purification without proteinase K of pathogenic PrP from scrapie-infected hamster brain. The Western blot analysis after SDS PAGE shows respectively equal amounts (1×10⁻³ gram equivalents) of the individual purification steps of NaPTA precipitation. BH: brain homogenizate; S: supernatant; W: supernatant from washing step; P: resulting pellet. Of every step, controls that have been treated by proteolysis with 5 μg/ml of proteinase K (PK) for one hour at 37° C. (+) are plotted. The purification was represented with scrapie-infected brain homogenizate (A) and with a non-infected brain sample (B).

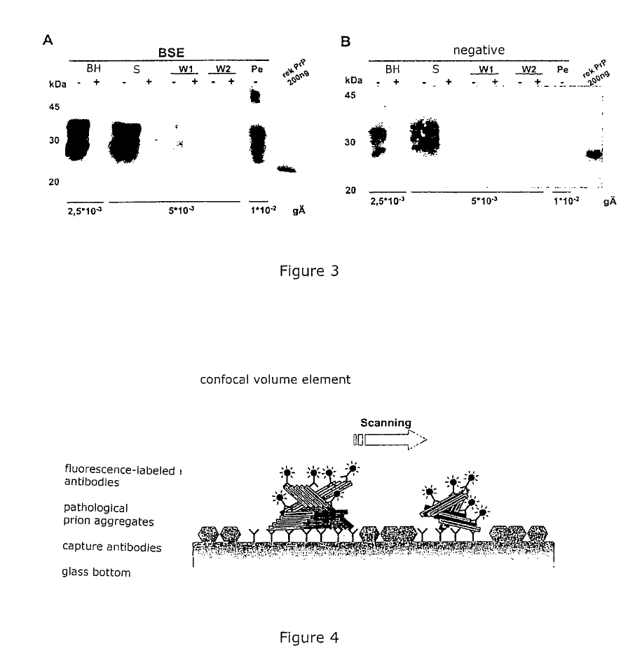

FIG. 3 shows a Western blot analysis of the NaPTA precipitation for the purification of pathogenic PrP^{BSE} from the medulla oblongata of a BSE-infected cattle. The Western blot analysis after SDS PAGE shows respective amounts (2.5×10⁻³ to 1×10⁻² gram equivalents) of the individual purification steps of NaPTA precipitation. The purification was performed with samples from a BSE-infected medulla oblongata of cattle (A) and a non-infected control (B). BH: brain homogenizate; S: supernatant; W1-2: supernatant from washing step; P: resulting pellet. Of every step, controls that have been treated by proteolysis with 5 μg/ml of proteinase K (PK) for one hour at 37° C. (+) are plotted. In both cases, 200 ng of bovine recombinant PrP(29-231) (rekPrP) is plotted as a control.

FIG. 4 shows a schematic representation of a single particle immunosorbent laser-scanning assay (SPILA).

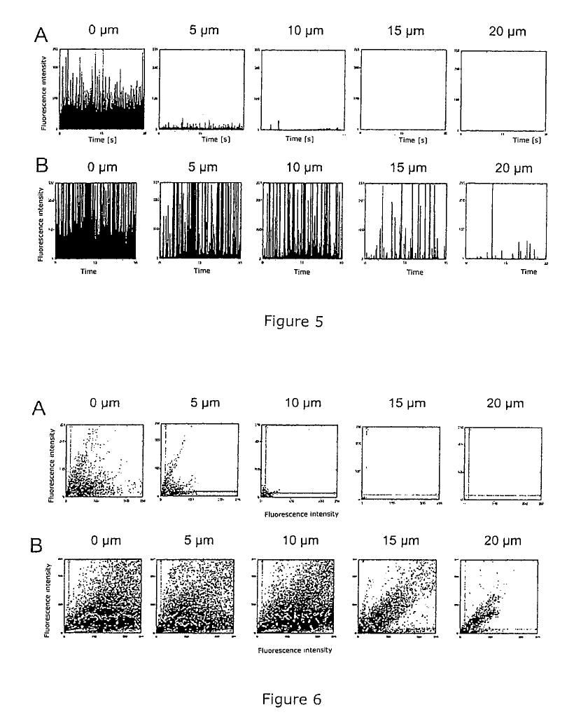

FIG. 5 shows fluorescence intensity measurements as a function of the height of the focus of the FCS optical system over the surface (“height scans”) of immobilized negSHa (A) and PrP^{Sc} (B) samples, respectively. Fluorescence-labeled (Alexa 633) D13 antibody (Inpro, USA) was employed as a probe for detection. The samples were measured at a distance of 0-20 μm from the chip surface in 5 μm steps for 30 seconds each in FCS.

FIG. 6 shows 2D-FIDA plots of fluorescence intensity measurements of immobilized negSHa (A) and PrP^{Sc} (B) samples, respectively. Fluorescence-labeled antibodies R1 and D13 (Alexa 488-labeled R1, Alexa 633-labeled D13; Inpro, USA) were employed as probes for detection. The samples were measured at a distance of 0-20 μm from the chip surface in 5 μm steps for 30 seconds each in FCS.

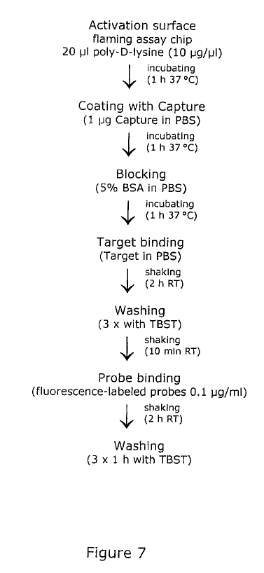

FIG. 7 schematically shows the steps of the protocol according to the invention for the immobilization and labeling of pathological prion aggregates.

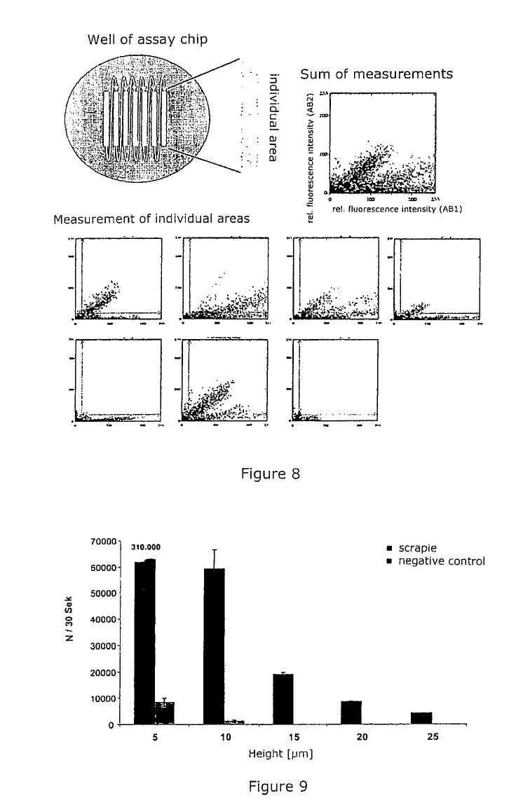

FIG. 8 shows the determination and summing of seven defined areas of an immobilized PrP^{Sc} sample. The measurement was performed at a distance of 15 μm above the chip surface. Fluorescence-labeled antibodies R1 and D13 (Alexa 488-labeled R1, Alexa 633-labeled D13; Inpro, USA) were employed as probes for detection. The measurements were performed for 30 seconds each on seven adjacent areas with a scanning movement. The results of the individual measurements were summed.

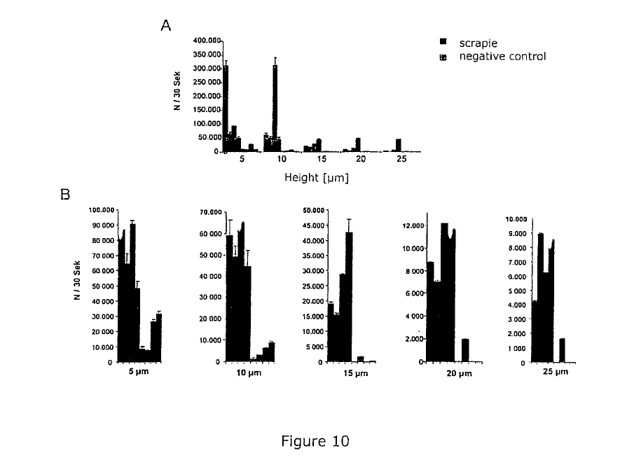

FIG. 9 shows the evaluation of 2D-FIDA measurements by an immobilized PrP^{Sc} sample (red) and a negative control (green). Antibody R1 was employed as the capture molecule, and the fluorescence-labeled anti-bodies R1 and D13 (Alexa 488-labeled R1, Alexa 633-labeled D13; Inpro, USA) were employed as detection probes. The samples were measured at a distance of 5-25 μm from the chip surface in 5 μm steps for 30 seconds each in FCS.

FIG. 10 shows the measurement (“height scan”) of each of four immobilized prp^{Sc} samples (red) and negative controls (green) by a 2D-FIDA measurement. Antibody R1 was employed as the capture molecule, and the fluorescence-labeled antibodies R1 and D13 (Alexa 488-labeled R1, Alexa 633-labeled D13; Inpro, USA) were employed as probes for detection. The samples were measured at a distance range of 5-25 μm from the chip surface in 5 μm steps for 30 seconds each in FCS. (A) Total of all measurements at the different distances from the chip surface, (B) representation of the measurements separated by heights of 5-25 μm with a zoomed ordinate, the respectively highest values being cut off.

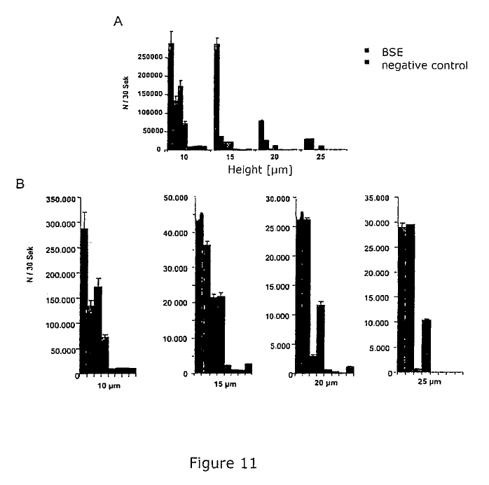

FIG. 11 shows a measurement (“height scan”) of each of four immobilized PrP^{BSE} samples (red) and negative controls (green) by a 2D-FIDA measurement. Antibody Saf32 was employed as the capture molecule, and the fluorescence-labeled antibodies 12F10 and Saf32 (Alexa 488-labeled 12F10, Alexa 633-labeled Saf32; Spibio, USA) were employed as detection probes. The samples were measured at a distance of 10-25 μm from the chip surface in 5 μm steps for 30 seconds each in FCS. (A) Total of all measurements at the different distances from the chip surface, (B) representation of the measurements separated by heights of 10-25 μm with a zoomed ordinate, the respectively highest values being cut off.

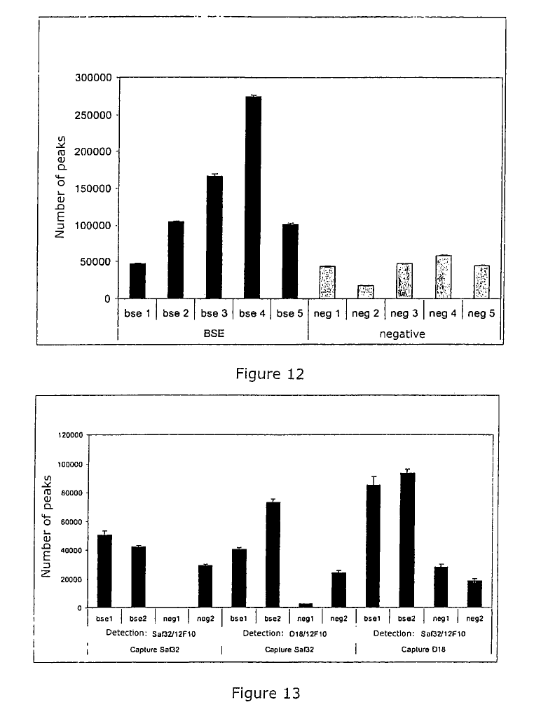

FIG. 12 shows a 2D-FIDA measurement of immobilized BSE cerebrospinal fluid samples and negative controls. The immobilization was performed with five PrP^{BSE} cerebrospinal fluid samples (bse 1-5) and five negative controls (neg 1-5). Antibody Saf32 was employed as the capture molecule, and the fluorescence-labeled antibodies 12F10 and Saf32 were employed as detection probes. The samples were measured at 5 μm above the surface of the glass bottom.

FIG. 13 shows a 2D-FIDA measurement of immobilized BSE cerebrospinal fluid samples and negative controls. The immobilization was performed with two PrP^{BSE} cerebrospinal fluid samples (bse 1 and bse 2) and two negative controls (neg 1 and neg 2). Antibodies Saf32 and D18 were employed as capture molecules, and the fluorescence-labeled antibodies 12F10, Saf32 and D18 were employed as detection probes in the respectively stated combinations. The samples were measured at 5 μm above the surface of the glass bottom.

### EXAMPLE 1

### Purification of PrP^{Sc} from Hamster Brain Tissue without Proteolysis

The purification of PrP^{Sc} (FIG. 1) was performed by analogy with a protocol by Safar et al. (Safar, J. et al. (1998) Nat. Med. 4, 1157-1165). Brain tissue from scrapie-infected Syrian hamsters and non-infected control samples were obtained from the RKI Berlin (Dr. M. Beekes) and the UCSF, San Francisco, USA (Dr. S. Prusiner). The brain tissue in PBS with 2% sarkosyl was processed into a 5% (w/v) homogenizate by means of a homogenizer (PowerGen 125, Fisher Scientific). This homogenizate was centrifuged at 5000×g for one minute to sediment larger tissue fragments. Subsequently, benzonase (Merck, Darmstadt, Germany) was added to a final concentration of 50 U/ml to degrade DNA and RNA (incubation with shaking for 45 minutes at 37° C.). Thereafter, NaPTA (final concentration 0.25%) and MgCl₂ (final concentration 10.6 mM) were added, and the precipitation mix was shaken at 37° C. over night. Subsequently, the sample was centrifuged at 14,000×g for 30 minutes, and the supernatant was discarded. The pellet was washed with 250 μl of PBS/250 mM EDTA for at least 30 minutes at 37° C. and again centrifuged at 14,000×g for 30 minutes. This washing step was repeated twice. The use of PBS/250 mM EDTA, pH 8, as washing buffer gave a significantly improved purification as compared to known washing buffers with only 50 mM EDTA as described by Wadsworth et al. (Wadsworth, J. D. et al. (2001) Lancet 358, 171-180) for the purification of PrP CD from human brain tissue.

A Western blot analysis of the purification steps of the NaPTA precipitation of scrapie-infected and non-infected brain samples is shown in FIG. 2. In the pellet fraction of the purification from brain tissue of the scrapie-infected hamster, a large amount of PrP could be detected. A comparison with the pellet fraction of the sample from the non-infected hamster, which did not contain any PrP, allows to conclude that the PrP of the scrapie-infected sample is exclusively pathogenic PrP. This is also indicated by the proteinase K resistance of the major part of the precipitated PrP. FIG. 2B shows that PrP^{C} remains in the supernatant almost completely.

Thus, it could be shown that major parts of the pathogenic PrP present could be selectively enriched and concentrated by the precipitation in contrast to the natural PrP^{C}. The individual washing steps did not cause a loss of pathogenic PrP.

### EXAMPLE 2

### Purification of PrP^{BSE} from the Medulla Oblongata of Cattle Brain without Proteolysis

BSE-infected tissue samples from the medulla oblongata of cattle as well as purified BSE samples or corresponding negative controls were obtained from the VLA Weighbridge, Great Britain (Dr. R. Jackmann) and from the Bundesforschungsanstalt für Viruskrankheiten der Tiere, Insel Riems. The purification of PrP^{BSE} from the medulla oblongata of cattle brain without proteolysis is basically the same as the above described PrP^{Sc} purification from hamster brain. Differences exist with respect to the sarkosyl concentration in the homogenization of the tissue. In contrast to hamster tissue, 4% sarkosyl was employed for cattle tissue (according to Safar et al. (2002) Nat. Biotechnol. 20, 1147-1150), because the purification efficiency and the PrP^{BSE} yield were significantly reduced for lower sarkosyl concentrations in the homogenizate. In addition, the duration of the precipitation was reduced to four hours. Two washing steps were required to achieve the degree of purity as necessary for FIDA measurements.

FIG. 3 shows a Western blot analysis of the individual purification steps of the precipitation for a tissue sample from a BSE-infected cattle and a control sample from medulla oblongata tissue. When the BSE brain homogenizate treated with proteinase K is compared with the untreated one (FIG. 3A), it becomes clear that the BSE-infected sample contained a low proportion of resistant PrP^{BSE}. In the negative control (FIG. 3B), it can be clearly seen that PrP^{C} completely remains in the supernatant, and the pellet is evidently free of PrP^{C}. In the BSE sample, low amount of PrP could be detected in the first washing step. A comparison of the pellet fractions of the BSE sample with the control sample allows to conclude that the PrP in the pellet from the BSE sample was pathogenic PrP^{BSE}. The fact that this sample has a low resistant PrP^{BSE} fraction also allows to conclude that this PrP is sensitive PrP^{BSE} for the major part thereof.

### EXAMPLE 3

### Resuspending of the Precipitated PrP Aggregates

To the PrP^{Sc/BSE} pellet from the NaPTA precipitation, 200 μl of PBS was added. Subsequently, the pellet was exposed to different ultrasonic conditions. The ultrasonication treatment was effected three times for two seconds by means of an ultrasonic needle probe (Sonificator Labsonic U, Braun Dissel, Melsungen, Germany). When the needle probe was employed, the ultrasonic probe was directly immersed into the buffer.

### EXAMPLE 4

### Development of a New Analytical Strategy by Immobilizing Pathological Prion Aggregates and Detection by Means of 2D-FIDA SPILA

By means of a SPILA (single particle immunosorbent laser-scanning assay), purified prion aggregates are immobilized on the surface of a measuring chip by means of a capture molecule (such as an antibody) to be subsequently detected (FIG. 4). Since the immobilization prevents a movement of the aggregates, the surface can be searched for aggregates by “scanning” (i.e., by moving the confocal volume element). Two advantages were to be utilized by the fixation of the aggregates. On the one hand, the fixation should contribute to the reproducibility of the results, and on the other hand, the aggregates should be concentrated. Further, washing steps can be performed, and the signal-to-noise ratio can be thus improved.

A precondition for the detection of immobilized single prion particles by measurements in FCS is the ability of the confocal volume element to be exactly focused to the height of the area to be scanned. For this reason, in cooperation with the manufacturer of instruments Evotec Technologies (Hamburg, Germany), the FCS instrument was extended by a piezo element, which enables the height to be adjusted with an accuracy of 100 nm independently of the motor control.

### EXAMPLE 5

### Coating of Glass Surfaces with Capture Molecule Proteins

In order to immobilize the aggregates specifically on the glass surface of the measuring chips employed, the glass surface must be previously coated with a capture molecule (e.g., an antibody). Methods for coating glass surfaces with proteins are known to the skilled person. For evaluating the measuring mix, an adhesive bonding of the capture molecule to a surface previously activated with poly-D-lysine was performed. The same experiment was performed with covalently bound antibodies. However, the further applications were performed with adhesively coated capture molecules. The efficiency of the activation of the glass surface can be enhanced by briefly flaming the glass surface. For the coating, the 24-well assay chips (Evotec, Hamburg, Germany) with a glass bottom were briefly flamed by means of a Bunsen burner. Subsequently, 20 μl of poly-D-lysine (10 μg/ml) in PBS was added to the wells of the assay chips and incubated at 37° C. for one hour (optionally at 4° C. over night). Subsequently, 1 μg each of capture molecule in PBS (in later experiments, the antibodies R1 (Inpro, USA) for scrapie samples and Saf32 (Spibio, USA) for BSE samples) was added to the wells and incubated at 37° C. for one hour. Subsequently, three washing steps with PBS were performed, and free binding sites were blocked by incubation with 5% (w/v) of BSA for one hour.

### EXAMPLE 6

### Immobilization of Pathological PrP Aggregates

For the specific immobilization of the pathological PrP aggregates, PrP-specific capture molecules were used. At first, the antibody R1 was employed as a capture molecule for purified PrP^{Sc} from the brain tissue of scrapie-infected hamsters and corresponding negative controls from non-infected animals. Thus, 1.25×10⁻³ gram equivalents each of purified and ultrasonically resuspended samples were added to the R1-coated wells and shaken at room temperature for three hours. Subsequently, washing with TBST was performed three times for 10 minutes. To be able to detect the immobilized prion particles by means of fluorescence measurement, the fluorescence-labeled antibodies R1 and D13 (Inpro, USA) were used at a concentration of 0.1 μg/ml in TBST and incubated for two hours with shaking. Finally, the mixture was washed three times with TBST for one hour each.

To evaluate the height ranges above the glass surface of the analytical chip in which the immobilized particles were present, measurements were performed in FCS in z direction at first. Subsequently, comparative fluorescence measurements were performed with PrP^{Sc} samples and negative controls. The courses of fluorescence intensity show clear differences (FIG. 5). At heights of 5-20 μm above the glass surface, many fluorescence intensity peaks appeared in scrapie samples that did not occur in the negative control. These are due to the binding of fluorescence-labeled antibodies to the pathological prion aggregates.

The differences between scrapie-infected and non-infected samples became even clearer from the representation of the 2D-FIDA data (FIG. 6). The binding of the two fluorescence-labeled antibodies to immobilized PrP aggregates results in the appearance of a characteristic diagonal line of the fluorescence intensities in the 2D-FIDA plot. The x axis shows the fluorescence intensities of the antibody labeled with Alexa 488, and the y-axis shows the fluorescence intensities of the antibody labeled with Alexa 633. The frequencies of the occurrence of fluorescence intensities are coded in the z axis (shades of gray). Since enormously strong signals were measured, this characteristic correlation diagonal could be resolved only at a height of 15 μm.

The protocol according to the invention for immobilizing pathological prion aggregates followed by labeling with fluorescence-labeled antibodies as established herein is summarized in FIG. 7.

### EXAMPLE 7

### Optimization of the Measurement of Immobilized Protein Aggregates by Means of 2D-FIDA

To be able to cover aggregates on as large a surface area as possible, the measuring focus was not only determined as predefined by the FCS standard (“scanned”), but the surface area to be covered was extended. Thus, seven adjacent areas in a well of the assay chip (FIG. 8) were measured. A complete measurement of the bottom surface of a well has not been possible to date, since only chips with round wells were available and the midpoint of the well had to be set manually, so that a “safety margin” was necessary to prevent that a well was left during the measurement. As can be seen in the individual regulations, the distribution of aggregates over the various areas was not equal, as expected. However, by summing the areas, a representative description of the sample is obtained.

Using the established immobilization and measuring sample, a “height scan” through a scrapie sample and a negative control was performed. The measurements were evaluated by summing frequencies of the fluorescence intensities of above 15 μm. The results of the 2D-FIDA measurements show that a significant difference can be measured between the PrP^{Sc} sample and the negative control (FIG. 9).

### EXAMPLE 8

### 2D-FIDA Serial Measurements of PrP^{Sc} and PrP^{BSE}

In order to evaluate the sample to be measured with respect to its diagnostic applicability, serial measurements were performed with samples from four different infected and four non-infected animals as controls.

Thus, brain tissue samples from scrapie-infected hamsters and negative controls were purified by means of NaPTA precipitation, and 1.25×10⁻³ gram equivalents were immobilized on the assay chips with antibody R1 as a capture molecule after resuspension by means 0f 3×2 seconds of ultrasonication. For detection, 0.1 μg/ml each of fluorescence-labeled antibody R1 and D13 was employed as probes. After three one-hour washing steps with TBST, fluorescence measurements were performed in FCS of seven individual areas. The measurements were performed at different heights over the glass bottom. The results (FIG. 10) show that a significant difference between scrapie-infected and non-infected hamsters can be observed in the height ranges of 5 μm to 25 μm. The measuring height of from 10 μm to 20 μm was found to be a suitable measuring height, because above this distance, the positive signals significantly drop, and below 10 μm, the measuring background is increased by non-specific antibody binding and free dye molecules.

In summary, it may be said that in the hamster model, scrapie-infected hamsters could be distinguished significantly from non-infected hamsters, and in comparison with the measuring mixture in suspension, not only were the differences between positive and negative samples clearer, but above all, the standard deviations could be enormously reduced.

The system was subsequently adapted for BSE samples. Thus, the antibody 12F10 (Spibio) was employed as a capture molecule. All the other steps are effected by analogy with those of the above described scrapie system. The fluorescence-labeled antibodies 12F10 and Saf32 (Spibio, USA) were employed as probes for detection.

The results for four BSE-infected animals and four negative controls are shown in FIG. 11. The measurement shows that in a distance range of from 10 μm to 25 μm from the chip surface, a significant difference between BSE-infected and non-infected animals could be seen in all cases. The range from 10 μm to 15 μm was the most suitable measuring range since the positive signal decreased strongly in some samples from 20 μm.

By these serial measurements, it could be shown that SPILA is suitable for a diagnostic assay for the detection of BSE and for scrapie.

### EXAMPLE 9

### 2D-FIDA Measurements of Immobilized BSE Cerebrospinal Fluid Samples from Cattle

The process according to the invention was also adapted to the use of cerebrospinal fluid as the sample material.

Thus, a serial measurement with five samples was performed. The glass surface of the assay chips was coated with the corresponding capture molecule (here: Saf32) as described in Example 5. Then, 20 μl of pure cerebrospinal fluid from terminally BSE-afflicted cattle as well as cerebrospinal fluid from healthy cattle as negative controls were employed. The pure cerebrospinal fluid was subjected to ultrasonication three times for two seconds each before being used, then it was added to the coated assay chips, shaken at room temperature for two hours for binding and subsequently incubated at 4° C. over night. The further treatment and measurement was effected according to the protocol as already described in Examples 6-8. The fluorescence-labeled antibodies Saf32 (Spibio) and 12F10 and were employed as detection probes. The result of the measurements at a height of 5 μm above the bottom of the assay chips is shown in FIG. 12.

In this serial measurement, four BSE samples could be distinguished from the negative controls. Only one BSE sample exhibited a signal that was not higher than the background.

To optimize the preparation, possible combinations of capture molecule/detection probes were tested at first. Thus, the antibodies Saf32 (Spibio) and D18 (Inpro) as capture molecules were combined with the fluorescence-labeled detection probes antibody Saf32 (Spibio), 12F10 (Spibio) and D18 (Inpro). The measuring processes were performed with two BSE cerebrospinal fluid samples and two negative controls in a double experiment. The measuring process was performed as described in the first serial measurement. The result of the serial measurement at a height of 5 μm above the bottom of the assay chip is shown in FIG. 13. The serial measurement shows that a distinction between the BSE samples and the negative controls is possible with all combinations. The combination of D18 as a capture molecule and Saf32/12F10 as detection probes offers the most promising results, since the variability between the samples of one group is lowest, and a clear distinction can be made.

Therefore, the process according to the invention is suitable for diagnosing BSE from a cerebrospinal fluid measuring sample.

1. A method for the selective determination of the presence or amount of pathological protein deposits, comprising:
(a) immobilizing on a surface a capture molecule having specific binding affinity for substructures of the protein deposits to be determined;
(b) contacting the immobilized capture molecule with a sample to be measured that is suspected of containing pathological protein deposits or substructures thereof;
(c) incubating the immobilized capture molecule and sample of (b) to allow a complex to be formed from the immobilized capture molecule and said substructures of the protein deposits to be determined;
(d) contacting the resulting complex with at least one detectable unit having specific binding affinity for said substructures of the protein deposits to be determined and producing an optically detectable signal, wherein at least one of said at least one detectable unit produces a signal detectable by means of spectroscopic methods; and
(e) detecting the complex formation by measuring the overall signal produced by said at least one detectable unit.

2. The method according to claim 1, wherein said substructures of the pathological protein deposits to be determined comprise monomeric or oligomeric units of the protein deposits or fragments thereof.

3. The method according to claim 1, wherein said protein deposits are associated with neurodegenerative diseases.

4. The method according to claim 3, wherein said neurodegenerative diseases are selected from the group consisting of transmissible spongiform encephalopathies, Alzheimer's disease, Parkinson's disease, Huntington's chorea and hereditary cerebral amyloid angiopathy.

5. The method according to claim 4, wherein said neurodegenerative diseases are selected from the group consisting of Creutzfeldt-Jakob disease, scrapie and bovine spongiform encephalopathy.

6. The method according to claim 1, which further comprises the activation of the surface before the capture molecule is immobilized thereon.

7. The method according to claim 1, wherein said capture molecule is selected from the group consisting of monoclonal antibodies, polyclonal antibodies and antibody fragments.

8. The method according to claim 1, wherein said capture molecule consists of substructures of pathological protein deposits.

9. The method according to claim 1, wherein said sample to be measured is derived from a body fluid or tissue.

10. The method according to claim 9, wherein said body fluid is selected from the group consisting of cerebrospinal fluid, lymph, blood, urine and sputum.

11. The method according to claim 9, wherein said tissue is brain tissue.

12. The method according to claim 1, wherein said sample to be measured is purified before being contacted with the immobilized capture molecule.

13. The method according to claim 12, wherein said purification includes phosphotungstate precipitation without adding a proteinase.

14. The method according to claim 1, wherein two detectable units bind simultaneously to the complex formed from the immobilized capture molecule and the substructures of the protein deposits.

15. The method according to claim 14, wherein the signals from the two or more detectable units are determined by coincidence measurement.

16. The method according to claim 1, wherein said at least one detectable unit comprises a protein or polypeptide.

17. The method according to claim 16, wherein said protein or polypeptide is selected from the group consisting of monoclonal antibodies, polyclonal antibodies and antibody fragments.

18. The method according to claim 1, wherein the optically detectable signal produced by said at least one detectable unit is selected from the group consisting of absorption, fluorescence emission, chemiluminescence emission and bioluminescence emission.

19. The method according to claim 1, wherein the spectroscopic detection methods are selected from the group consisting of confocal fluorescence spectroscopy, fluorescence correlation spectroscopy (FCS), and FCS in combination with cross-correlation and single particle immunosorbent laser-scanning assay.

20. The method according to claim 19, wherein a fluorescence intensity distribution analysis is performed for evaluating the results.

21. The method according to claim 1, wherein the overall signal produced by said at least one detectable unit is measured by scanning the surface.

22. The method according to claim 21, wherein a plurality of adjacent subareas of the surface are scanned, and the individual values are subsequently added.
